(19) United States
(12) Patent Application Publication
(10) Pub. No.: US 2004/0260084 A1
(43) Pub. Date: Dec. 23, 2004

(54) Novel c-3 s/o-and s/n formaldehe acetal derivatives of cephalosporins and their use as antibotics

(76) Inventors: Hans R. Pfaendler, Stockdorf (DE); Wolfgang Jenni, Munchen (DE)

Correspondence Address: DAVID A. GUERRA, 933 OLANDER WAY SOUTH, SUITE 3, SOUTH PASADENA, FL, 33707, US

(21) Appl. No.: 10/490,927

(22) PCT Filed: Nov. 4, 2002
(86) PCT No.: PCT/EP02/12257

(30) Foreign Application Priority Data
Nov. 12, 2001 (EP) 01 12 6490.0

(51) Int. Cl. C07D 501/14

(52) U.S. Cl. 540/222

(57) ABSTRACT

The compounds of the general formula (I) wherein R¹ denotes a pharmaceutically acceptable side chain radical as used conventionally in the field of cephalosporins and wherein R² denotes a pharmaceutically acceptable group which is bonded to the remaining part of the molecule by an oxygen-carbon single bond or a nitrogen-carbon single bond, and their pharmaceutically acceptable salts, esters and amide derivatives are effective antibiotics.

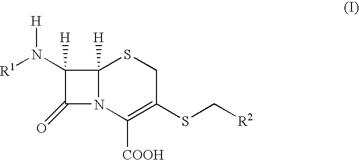

### DESCRIPTION AND BACKGROUND OF INVENTION

This invention relates to novel 3-S/O— and 3-S/N formaldehyde acetal derivatives of cephalosporins of the general formula I

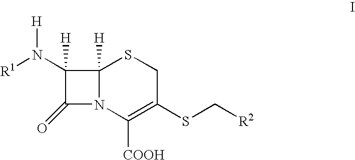

or a pharmaceutically acceptable salt, ester or amide derivative thereof wherein R¹ denotes a pharmaceutically acceptable side chain radical as used conventionally in the field of cephalosporins and wherein R² denotes a pharmaceutically acceptable group which is bonded to the remaining part of the molecule by an oxygen-carbon single bond or a nitrogen-carbon single bond.

More specifically, the invention relates to compounds of the formula I wherein R¹ denotes pharmaceutically acceptable side chain radicals selected from phenylacetyl, phenoxyacetyl, 2-amino-2-phenylacetyl, 2-amino-2-(4-hydroxyphenyl)acetyl, 2-amino-2-(1,4-cyclohexadienyl)acetyl, 2-hydroxy-2-phenylacetyl, 2-hydroxy-2-(4-hydroxyphenyl)acetyl, Z-2-(2-amino-4-thiazolyl)-2-(methoximino)acetyl, Z-2-(2-amino-4-thiazolyl)-2,2-difluoromethoximino)-acetyl, Z-2-(2-amino-4-thiazolyl)-2-(carboxymethoxyimino)acetyl, Z-2-(2-amino-4-thiazolyl)-2-((1-carboxy-1-(methylethoxy)imino)acetyl, Z-2-(2-amino-4-thiazolyl)-2-(hydroxyimino)acetyl, Z-2-(2-aminothiazol4-yl)-2-pentenoyl, 2-(2-amino-4-thiazolyl)₄-carboxy-1-oxo-2-butenyl, 2-(cyanomethylthio)acetyl, 2-(difluoromethylthio)acetyl, 2-(fluoromethylthio)acetyl, 2-(2-amino-2-carboxyethylthio)acetyl, α-(4-ethyl-2,3-dioxo-1-piperazinecarboxamido)-α-(4-hydroxyphenyl)acetyl, 2-(2-(aminomethyl)phenyl)acetyl, [4-(2-amino-1-carboxy-2-oxoethylidene)-1,3-dietan-2-yl]carbonyl, 2-thienylacetyl, 1-(1H)-tetrazolylacetyl, 2-(3,5-dichloro-4-pyridon-1-yl)acetyl, 2-(5-carboxy4-imidazolylcarboxamido)phenylacetyl, phenylsulfoacetyl, 2-furanyl(methoxyimino)acetyl, cyanoacetyl, 5-amino-5-carboxy-1-oxopentyl, 2-(4-pyridylthio)acetyl, 5-amino-1,2,4-thiadiazol-3-yl(methoximino)acetyl, 1H-pyrazol-3-yl(methoximino)acetyl, and wherein R² denotes a pharmaceutically acceptable group which is bonded to the remaining part of the molecule by an oxygen-carbon single bond or a nitrogen-carbon single bond and which is selected from the group comprising substituted or unsubstituted: alkoxy, alkenyloxy, alkinyloxy, cycloalkoxy, N-heterocyclyl, heterocyclyloxy, heterocyclylcarbonyloxy, heterocyclylthiocarbonyloxy, acyloxy, thioacyloxy, alkoxycarbonyloxy, carbamoyloxy, thiocarbamoyloxy, heterocyclyloxycarbonyloxy, heterocyclyloxythiocarbonyloxy, N-heterocyclycarbamoyloxy, N-heterocyclylthiocarbamoyloxy, heterocyclylcarbonylamino, heterocyclylthiocarbonylamino, heterocyclyloxycarbonylamino, acylamino, alkoxycarbonylamino, alkoxythiocarbonylamino, thioacyclamino, N-heterocyclylcarbamoylamino, N-heterocyclylthiocarbamoylamino, carbamoylamino, thiocarbamoylamino, imidoylamino, guanidino, N-heterocyclyl-alkoxycarbonylamino, N-heterocyclyl-alkylthiocarbonylamino and N-sulfonylamino where the foregoing alkyl, alkenyl, alkinyl, acyl, thioacyl or imidoyl molecule parts contain 1 to 6 carbon atoms and the heterocyclyl moiety is monocyclic or bicyclic and contains 3 to 10 ring atoms, of which one or more are selected from the series comprising: oxygen, sulphur or nitrogen and where the substituents of the above-mentioned groups R², independently of one another, may be: alkyl, acyl, thioacyl, heterocyclyl, hydroxyl, hydroxyalkyl, alkoxy, hydroxyalkoxy, aminoalkoxy, amidinoalkoxy, guanidinoalkoxy, acyloxy, heterocyclyloxy, alkylheterocyclyloxy, hydroxyalkylheterocyclyloxy, aminoalkylheterocyclyloxy, carbamoyl, alkylcarbamoyl, dialkylcarbamoyl, carbamoyloxy, alkylcarbamoyloxy, dialkylcarbamoyloxy, thiocarbamoyl, alkylthiocarbamoyl, dialkylthiocarbamoyl, thiocarbamoyloxy, alkylthiocarbamoyloxy, dialkylthiocarbamoyloxy, mercapto, alkylthio, hydroxyalkylthio, aminoalkylthio, monoalkylaminoalkylthio, dialkylaminoalkylthio, amidinoalkylthio, acylthio, heterocyclylthio, alkylheterocyclylthio, hydroxyalkylheterocyclylthio, aminoalkylheterocyclylthio, carbamoylthio, monoalkylcarbamoylthio, dialkylcarbamoylthio, thiocarbamoylthio, alkylthiocarbamoylthio, dialkylcarbamoylthio, amino, monoalkylamino, hydroxyalkylamino, aminoalkylamino, dialkylamino, oxo, oximino, or alkylimino, imidoylamino, alkylimidoylamino, dialkylimidoylamino, tetraalkylammonium, cycloalkylamino, heterocyclylamino, alkylheterocyclylamino, heterocyclylcarbonylamino, alkylheterocyclylcarbonylamino, acylamino, amidino, monoalkylamidino, dialkylamidino, guanidino, alkylguanidino, dialkylguanidino, carbamoylamino, thiocarbamoylamino, alkylcarbamoylamino, thiocarbamoylamino, alkylthiocarbamoylamino, nitro, chloro, bromo, fluoro, iodio, azido, cyano, alkylsulphinyl, alkylsulphonyl, sulphonamido, sulphamoyloxy, alkylsulphamoyloxy, alkylsulphonyloxy or sulpho, sulphoxy, carboxamido, N-monoalkylcarboxamido, N,N-dialkylcarboxamido or carboxy, where the substituents, independently of one another, occur once or several times and their alkyl moiety contains 1 to 6 carbon atoms, and where the heterocyclic moiety is monocyclic or bicyclic and contains 3 to 10 ring atoms, of which one or more are selected from the series comprising: oxygen, sulphur and nitrogen, which compounds and their pharmaceutically acceptable salts, esters and amide derivatives are useful as antibiotics.

Pharmaceutically acceptable groups R³, which are bonded via an oxygen-carbon single bond or a nitrogen-carbon single bond are groups as are customary, for example, in the field β-lactam antibiotics or β-lactamase inhibitors. Such groups are found, for example, in M. S. Sassiver, A. Lewis in “Advances in Applied Microbiology”, Ed. D. Perlman, Academic Press N.Y. (1970) or in many patents, e.g. U.S. Pat. No. 5,096,899.

The term “pharmaceutically acceptable salt” as used herein and in the claims, includes non-toxic acid and base salts and the salts of zwitterionic species. Salts with a base include inorganic salts such as sodium, potassium, magnesium and calcium, or ammonium and salts with non-toxic amines such as trialkylamines, alkanolamines, arginine or cyclic amines such as piperazine, procaine and other amines, which have been used to form salts of carboxylic acids. Salts with an acid include inorganic acid salts such as hydrochloride, sulfate, phosphate and the like and organic acid salts such as acetate, maleate, citrate, succinate, ascorbate, lactate, fumarate, tartrate and oxalate and other organic salts with acids which have been used to form salts with amines.

The term “pharmaceutically acceptable side chain radicals” includes groups known in the art, for example from the numerous side chains of penicillins or cephalosporins published in Advances in Drug Res. 17, 146-164 (1988).

The pharmaceutically acceptable esters and amide derivatives as used herein, serve as prodrugs by being hydrolyzed in the body to yield the antibiotic per se. They are preferably administered orally since hydrolysis occurs principally under the influence of the digestive enzymes. Parenteral administration may be used in some instances where hydrolysis occurs in the blood. Examples of pharmaceutically acceptable esters and amide derivatives include physiologically hydrolyzable esters and amides known and used in the penicillin and cephalosporin fields as, e.g. in Advances in Drug Res. 17, 197-203 (1988). Such esters and amide derivatives are prepared by conventional techniques known in the art.

The compounds according to the invention have several asymmetric centers and can thus exist in in several stereochemical forms. The invention includes the mixture of isomers and the individual stereoisomers. The most preferred compounds of formula I have the configuration 6R and 7R of the natural Cephalosporin C in accordance with that of many commercially available cephalosporins such as Cefaclor, Cefdinir, Cefepime, Cefixime, Cefotaxime and the like. The formulae and configuration of these and many related compounds are depicted in Merck Index, Vol. 12.

This invention also relates to processes for the preparation of compounds (I), pharmaceutical compositions comprising such compounds and to methods of treatment comprising administering such compounds and compositions when an antibiotic effect is indicated.

The terminology for compounds of this class may either be based upon the root name “cephem” which employs a trivial and simple system of nomenclature (used in the general description). Alternatively, these compounds can also be described by the nomenclature according to the Chemical Abstract system (bicyclo-nomenclature) which is more appropriate to describe individual compounds of this family.

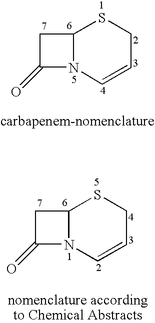

Therefore the Chemical Abstract nomenclature is used within the Example Section.

The cephalosporins belong to the most important antibiotics. Although new generations of β-lactams such as penems or carbapenems which have a higher in vitro-activity have been developed very recently, the cephalosporins kept their position in the market. This is particularly due to their extremely low toxicity as compared to many other antibiotics. Typical LD₅₀ exceed 5000 mg/kg. Another important advantage is their higher stability in blood serum, leading to higher blood levels than those achieved with the mentioned nonclassical β-lactams. This is true in particular with the injectable third generation cephalosporins, e.g with Ceftriaxone or Ceftazidime, having also sufficient activity against many penicillin resistant bacteria.

LD₅₀ values for many cephalosporins are given in Merck Index, 12th ed., the serum stabilities of cephalosporins and carbapenems are published in D. Milatovic, I. Braveny, “Infektionen”, 6^{th} ed. Vieweg, Braunschweig, 1997.

Third generation cephalosporins have also been developed for oral application. They have never reached the position of older oral penicillins or cephalosporins in the market. As compared to older oral cephalosporins such as Cephalexin or Cefaclor the newer cephalosporins are relatively poorly resorbed. As a consequence the non-resorbed antibiotics remain in the colon and interfere with the bacterial flora, resulting in substantial adverse side effets. A detailed description of this problem is given in Moellering, R. C. Jr. (ed.) “Oral Cephalosporins”, Antibiot. Chemother.,Vol. 47 p.1-7, 72-109, 161-181 Basel, Karger 1995.

Consequently, there is a continuing need for new antibiotics. This search is particularly acute for β-lactams which are stable in human blood serum and against bacterial resistance enzymes such as l-lactamases, for example TEM 1 β-lactamase, and which are well resorbed by the oral route.

Therefore it is a objective of the present invention to provide a novel class of cephalosporins with high blood serum stability and also with stablity to β-lactamases and being absorbed by the oral route.

The compounds of the above-mentioned formula I are conveniently prepared in accordance with the following equation:

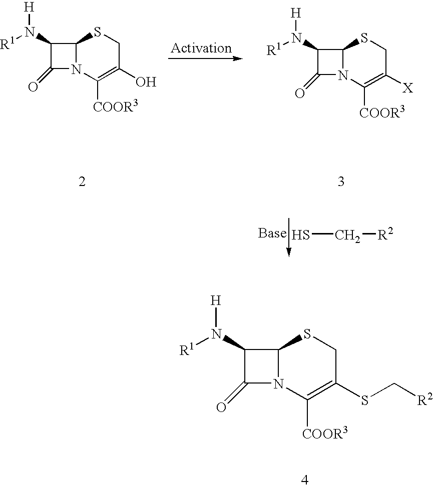

In the above-mentioned equation a 3-hydroxy-cephem ester 2 is converted into an activated enol with an activating agent and an inorganic or organic base leading to activated enol 3. Examples for such activating agents are trifluoromethanesulfonic anhydride or diphenylphosphoroyl chloride and the like. Examples or inorganic bases are sodium hydroxide or potassium carbonate and the like. They are preferably used neat or in aqueous solutions by the phase transfer technique. Preferred solvents are tetrahydrofuran or acetonitrile or methylene chloride-water mixtures. Useful organic bases are triethylamine or N-diisopropylethylamine and the like. With organic bases the reaction is carried out in organic solvents, preferably methylene chloride.

3-Hydroxy-cephem esters 2 are known in the art and described, for example in S. Kukoija et al. Journ. Amer. Chem. Soc. 1976, 98, 5040-5041 or in H. Tanaka et al. Bull. Chem. Soc. Jpn. 1995, 68, 1385-1391. The ester protecting group R³ is a removable group which is known in the art. Examples are 2,2,2-trichloroethyl, p-nitrobenzyl or preferably benzhydryl.

The acyl group R¹ in the above-mentioned 3-hydroxycephem esters 2 is not very critical for the following reactions. It is preferred to use R¹=PhCH₂CO or R¹=PhOCH₂CO since these residues are derived from the side chains of the natural penicillins G or V.

The activated enol 3 is then converted into the S/O or S/N formaldehyde acetal derivative 4 using thiols HS—CH₂—R² and an organic base, for example triethylamine or N,N-diisopropylethylamine. The preparation of the thiols and the substitution reaction is known in the art and has recently been disclosed in the field of carbapenems (PCT EP 99 05 295, Publ. No. WO 00 05 574 A). This process is preferably carried out at low temperature, preferably −78° C. with slow addition of the base in order to avoid double bond migration of the 3-cephem.

Because of the high reactivity of the above-mentioned HS/O- or HS/N-formaldehyde acetals the reaction temperature can be varied within a large range. Preferably the process 3→4 is carried out between −70° C. and room temperature. A unpolar or polar solvent such as methylene chloride or acetonitrile, or preferably N,N-dimethylformamide, is suitable. The process 3→4 can also be carded out using phase transfer conditions, for example those using water, an unpolar solvent such as carbon tetrachloride or methylene chloride and a phase transfer catalyst such as tetrabutylammonium bromide.

The process 3→4 can also be carried out by using a preformed salt, preferably an alkali, earth alkali or tetraalkylammonium salt of the HS/O- or HS/N-formaldehyde hemiacetals. With the inorganic salts, the process 34 is preferably carried out without additional base in a polar solvent, for example N,N-dimethylformamide. With the more soluble tetraalkylammonium salts, a less polar solvent such as tetrahydrofuran is preferable.

In a preferred one pot process the activated enol 3 is not isolated but converted in situ to the S/O or S/N formaldehyde acetal derivative 4.

The HS/O- and HS/N-formaldehyde hemiacetals HS—CH₂—R² are two classes of compounds discovered and described only very recently in PCT EP 99 05 295, Publ. No. WO OO O5 574 A). They are a prerequisite for the preparation of the cephalosporins according to the invention. As these reagents were not disclosed prior to 2000 the novel C-3 S/O— and S/N formaldehyde acetal derivatives of cephalosporins of formula I according to the invention have not been prepared earlier and never appeared in the literature.

The acyl group R¹ is then removed in a three step sequence. The amido group of 4 is converted first into the imido chloride 5 with a inorganic acid chloride, preferaby with PCl₅ and an organic base such as morpholine, triethylamine, pyridine and the like, preferably with N,N-dimethylaniline in an organic solvent, preferably methylene chroride.

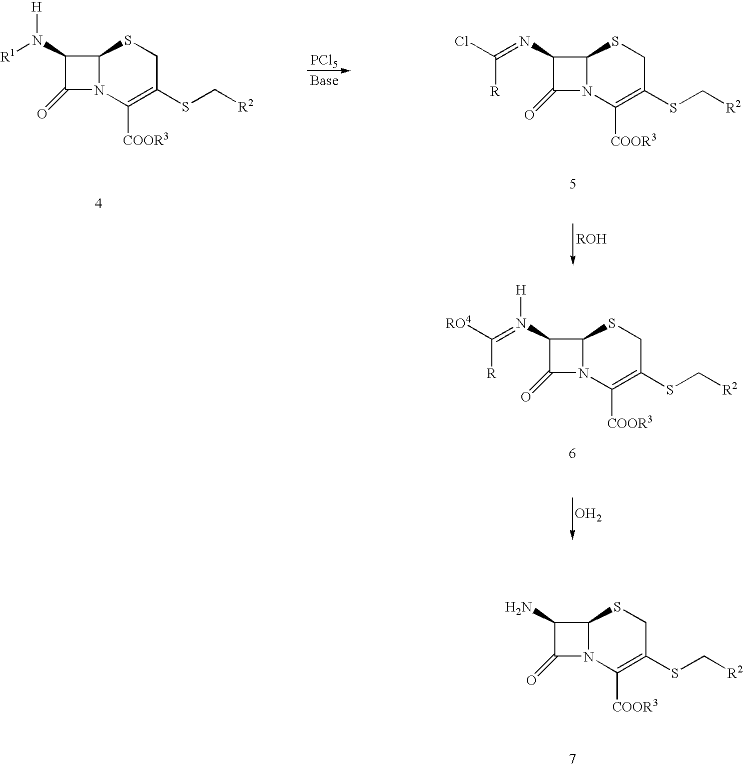

The imino chloride 5 is then converted into imino ether 6 using a dry alcohol such as isobutanol, isopropanol or ethanol at low temperature. A preferred alcohol for this process is dry methanol, leading to precipitation of the pure hydrochloride of imino ether 6 at −200 C, thus facilitating the isolation.

The imino chlorides 5 and the imino ethers 6 can exist as a cis-trans isomeric mixture. The ratio of this mixture is not very critical as both stereoisomers can be converted to a single stereoisomer, i.e. the 7-amino-cephem 7, by hydrolysis.

Finally, the imino ether 6 or its hydrochloride is hydrolyzed with aqueous acid such as sulfuric acid or hydrochloric acid, affording the free 7-amino-cephem 7 after neutralisation with an inorganic base and extraction with an organic solvent, preferably ethyl acetate.

This three-step-procedure of removing a side chain of cephalosporins is known in the field and described, for example in G. I. Gregory, ed. Recent Adv. Chem. β-Lact. Antibiot., Special Publ. No. 38, p. 109-124, The Royal Soc., London 1980.

The removable ester group R³ can be removed from the 7-amino-cephem 7 to afford the unprotected zwitterionic nucleus 8. The reagents for this process depend on the character of the protecting group and are known in the art. For example a benzhydryl group R³ can be removed by using an excess of a strong organic or Lewis acid, preferably trifluoroacetic acid or aluminum trichloride and a scavenger, preferably methoxybenzene.

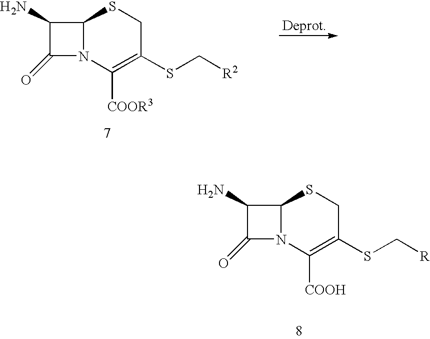

The protecting groups R³ in the starting materials 2 and in the described intermediates 3-8 are easily removable radicals which are known per se, as are usually used for the purpose in organic synthesis. Protection groups of this kind are found, for example, in Gunda I. Georg “The Organic Chemistry of β-Lactams” VCH Publishers UK, Cambridge, 1993, pp 1-29.

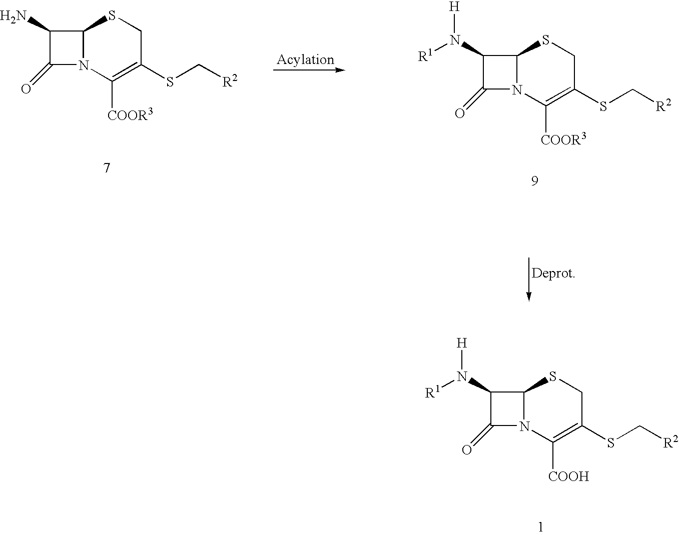

The acylation step 7→9 is carried out by a reaction of a protected or unprotected activated side chain carboxylic acid. By this process the side chains are incorporated into the 6-amino-cephems 7 or 8. The relevant side chain carboxylic acids R1-OH can be activated by methods known in the field of penicillins or cephalosporins. For example acid chlorides or mixed anhydrides of the side chain carboxylic acid with, for example, alkylcarbonic acids can be used. Another technique uses thioesters, for example, those derived from the side chain carboxylic acids R¹—OH and 2-mercaptobenzthiazol. The activated side chain carboxylic acids may contain protection groups, known in the art. A specially preferred version of the acylation uses activated carboxylic acids, protected with protection groups that are cleaved simultaneously with the ester protection group R³. Examples of this version include N-BOC-protecting groups in the side chains which are simultaneosly cleaved with the bezhydryl protecting group R³ by strong acids, for example trifuoroacetic acid or aluminum trichloride. This version of simultaneous deprotection is also exemplified in the Example Section of this patent application.

The acylation step, starting with esters 7, can be carried out in a variety of organic solvents, for example tetrahydrofuran, methylene chroride and the like in presence or without a base, for example triethylamine or pyridine at temperatures between −70° C. and room temperature. The acylation step can also be carried out using phase transfer conditions with mixtures of water and organic solvents, for example dichloromethane at temperatures between 0° C. and room temperature with inorganic bases, for example sodium bicarbonate.

The acylation step, starting with the zwitterionic 8, can be carried out in organic solvents or water or mixtures of organic solvents and water with bases, for example sodium bicarbonate.

An alternative consists in protecting the free carboxylic acid group of 8 with a silylating agent, for example N,O-bis(trimethylsilyl)acetamide and in acylating the intermediate silyl ester. The acylated silyl ester is then hydrolyzed in aqueous solution to afford the acylated cephalosporins 1.

Within the deprotection step 9→1, the free carboxylic acid or the corresponding inorganic or organic salts are formed. The reaction conditions and reagents used in the deprotection step 9→1 depend on the character of the ester protection group R³. Reagents for this step 9→1 are known in the art and described, for example in Gunda I. Georg “The Organic Chemistry of 13-lactams” VCH Publishers UK, Cambridge, 1993, pp. 1-29.

In the general description of the present invention, R1 denotes pharmaceutically acceptable side chain radicals as used conventionally in the field of cephalosporins and wherein R² denotes a pharmaceutically acceptable group which is bonded to the remaining part of the molecule by an oxygen-carbon single bond or a nitrogen-carbon single bond.

A preferred class of compounds I is that, in which R1 denotes pharmaceutically acceptable side chain radicals selected from phenylacetyl, phenoxyacetyl, 2-amino-2-phenylacetyl, 2-amino-2-(4-hydroxyphenyl)acetyl, 2-amino-2-(1,4-cyclohexadienyl)acetyl, 2-hydroxy-2-phenylacetyl, 2-hydroxy-2-(4-hydroxyphenyl)acetyl, Z-2-(2-amino-4-thiazolyl)-2-(methoximino)acetyl, Z-2-(2-amino-4-thiazolyl)-2,2-difluoromethoximino)-acetyl, Z-2-(2-amino-4-thiazolyl)-2-(carboxymethoxyimino)acetyl, Z-2-(2-amino-4-thiazolyl)-2-((1-carboxy-1-(methylethoxy)imino)acetyl, Z-2-(2-amino-4-thiazolyl)-2-(hydroxyimino)acetyl, Z-2-(2-aminothiazol4-yl)-2-pentenoyl, 2-(2-amino-4-thiazolyl)₄-carboxy-1-oxo-2-butenyl, 2-(cyanomethylthio)acetyl, 2-(difluoromethylthio)acetyl, 2-(fluoromethylthio)acetyl, 2-(2-amino-2-carboxyethylthio)acetyl, α-(4-ethyl-2,3-dioxo-1-piperazinecarboxamido)-α-(4-hydroxyphenyl)acetyl, 2-(2-(aminomethyl)phenyl)acetyl, [4-(2-amino-1-carboxy-2-oxoethylidene)-1,3-dietan-2-yl]carbonyl, 2-thienylacetyl, 1-(1H)-tetrazolylacetyl, 2-(3,5-dichloro-4-pyridon-1-yl)acetyl, 2-(5-carboxy4-imidazolylcarboxamido)phenylacetyl, phenylsulfoacetyl, 2-furanyl(methoxyimino)acetyl, cyanoacetyl, 5-amino-5-carboxy-1-oxopentyl, 2-(4-pyridylthio)acetyl, 5-amino-1,2,4-thiadiazol-3-yl(methoximino)acetyl, 1H-pyrazol-3-yl(methoximino)acetyl, and wherein R² denotes a pharmaceutically acceptable group which is bonded to the remaining part of the molecule by an oxygen-carbon single bond or a nitrogen-carbon single bond and which is selected from the group comprising substituted or unsubstituted: alkoxy, alkenyloxy, alkinyloxy, cycloalkoxy, N-heterocyclyl, heterocyclyloxy, heterocyclylcarbonyloxy, heterocyclylthiocarbonyloxy, acyloxy, thioacyloxy, alkoxycarbonyloxy, carbamoyloxy, thiocarbamoyloxy, heterocyclyloxycarbonyloxy, heterocyclyloxythiocarbonyloxy, N-heterocyclycarbamoyloxy, N-heterocyclylthiocarbamoyloxy, heterocyclylcarbonylamino, heterocyclylthiocarbonylamino, heterocyclyloxycarbonylamino, acylamino, alkoxycarbonylamino, alkoxythiocarbonylamino, thioacyclamino, N-heterocyclylcarbamoylamino, N-heterocyclylthiocarbamoylamino, carbamoylamino, thiocarbamoylamino, imidoylamino, guanidino, N-heterocyclyl-alkoxycarbonylamino, N-heterocyclyl-alkylthiocarbonylamino and N-sulfonylamino where the foregoing alkyl, alkenyl, alkinyl, acyl, thioacyl or imidoyl molecule parts contain 1 to 6 carbon atoms and the heterocyclyl moiety is monocyclic or bicyclic and contains 3 to 10 ring atoms, of which one or more are selected from the series comprising: oxygen, sulphur or nitrogen and where the substituents of the above-mentioned groups R², independently of one another, may be: alkyl, acyl, thioacyl, heterocyclyl, hydroxyl, hydroxyalkyl, alkoxy, hydroxyalkoxy, aminoalkoxy, amidinoalkoxy, guanidinoalkoxy, acyloxy, heterocyclyloxy, alkylheterocyclyloxy, hydroxyalkylheterocyclyloxy, aminoalkylheterocyclyloxy, carbamoyl, alkylcarbamoyl, dialkylcarbamoyl, carbamoyloxy, alkylcarbamoyloxy, dialkylcarbamoyloxy, thiocarbamoyl, alkylthiocarbamoyl, dialkylthiocarbamoyl, thiocarbamoyloxy, alkylthiocarbamoyloxy, dialkylthiocarbamoyloxy, mercapto, alkylthio, hydroxyalkylthio, aminoalkylthio, monoalkylaminoalkylthio, dialkylaminoalkylthio, amidinoalkylthio, acylthio, heterocyclylthio, alkylheterocyclylthio, hydroxyalkylheterocyclylthio, aminoalkylheterocyclylthio, carbamoylthio, monoalkylcarbamoylthio, dialkylcarbamoylthio, thiocarbamoylthio, alkylthiocarbamoylthio, dialkylcarbamoylthio, amino, monoalkylamino, hydroxyalkylamino, aminoalkylamino, dialkylamino, oxo, oximino, or alkylimino, imidoylamino, alkylimidoylamino, dialkylimidoylamino, tetraalkylammonium, cycloalkylamino, heterocyclylamino, alkylheterocyclylamino, heterocyclylcarbonylamino, alkylheterocyclylcarbonylamino, acylamino, amidino, monoalkylamidino, dialkylamidino, guanidino, alkylguanidino, dialkylguanidino, carbamoylamino, thiocarbamoylamino, alkylcarbamoylamino, thiocarbamoylamino, alkylthiocarbamoylamino, nitro, chloro, bromo, fluoro, iodio, azido, cyano, alkylsulphinyl, alkylsulphonyl, sulphonamido, sulphamoyloxy, alkylsulphamoyloxy, alkylsulphonyloxy or sulpho, sulphoxy, carboxamido, N-monoalkylcarboxamido, N,N-dialkylcarboxamido or carboxy, where the substituents, independently of one another, occur once or several times and their alkyl moiety contains 1 to 6 carbon atoms, and where the heterocyclic moiety is monocyclic or bicyclic and contains 3 to 10 ring atoms, of which one or more are selected from the series comprising: oxygen, sulphur and nitrogen, which compounds and their pharmaceutically acceptable salts, esters and amide derivatives are useful as antibiotics.

An especially preferred class of compounds I is that, in which the group R¹ denotes pharmaceutically acceptable side chain radicals selected from phenylacetyl, phenoxyacetyl, 2-amino-2-phenylacetyl, 2-amino-2-(4-hydroxyphenyl)acetyl, 2-amino-2-(1,4-cyclohexadienyl)acetyl, 2-hydroxy-2-phenylacetyl, 2-hydroxy-2-(4-hydroxyphenyl)acetyl, Z-2-(2-amino-4-thiazolyl)-2-(methoximino)acetyl, Z-2-(2-amino-4-thiazolyl)-2,2-difluoromethoximino)-acetyl, Z-2-(2-amino-4-thiazolyl)-2-(carboxymethoxyimino)acetyl, Z-2-(2-amino-4-thiazolyl)-2-((1-carboxy-1-(methylethoxy)imino)acetyl, Z-2-(2-amino-4-thiazolyl)-2-(hydroxyimino)acetyl, Z-2-(2-aminothiazol4-yl)-2-pentenoyl, 2-(2-amino-4-thiazolyl)₄-carboxy-1-oxo-2-butenyl, 2-(cyanomethylthio)acetyl, 2-(difluoromethylthio)acetyl, 2-(fluoromethylthio)acetyl, 2-(2-amino-2-carboxyethylthio)acetyl, c-(4-ethyl-2,3-dioxo-1-piperazinecarboxamido)-(4-hydroxyphenyl)acetyl, 2-(2-(aminomethyl)phenyl)acetyl, [4-(2-amino-1-carboxy-2-oxoethylidene)-1,3-dietan-2-yl]carbonyl, 2-thienylacetyl, 1-(1H)-tetrazolylacetyl, 2-(3,5-dichloro-4-pyridon-1-yl)acetyl, 2-(5-carboxy-4-imidazolylcarboxamido)phenylacetyl, phenylsulfoacetyl, 2-furanyl(methoxyimino)acetyl, cyanoacetyl, 5-amino-5-carboxy-1-oxopentyl, 2-(4-pyridylthio)acetyl, 5-amino-1,2,4-thiadiazol-3-yl(methoximino)acetyl, 1H-pyrazol-3-yl(methoximino)acetyl, and wherein R² denotes a pharmaceutically acceptable group which is bonded to the remaining part of the molecule by an oxygen-carbon single bond or a nitrogen-carbon single bond and which is selected from the group methoxy and acetylamino.

The groups R¹ in the cephalosporin derivatives I according to the invention have a strong impact on the antibacterial activity. It is known in the art and described, predominantly in the field of parenteral cephalosporins, for example from Advances of Drug Res. 17, 61-234,1988, that a great variety of such side chains leads to high antibacterial activity and high (1-lactamase stability.

A selection of compounds I according to the invention showed high in-vitro antibacterial activity against various gram-positive and gram-negative bacteria. Surprisingly, the minimal inhibitory concentrations are practically independent on the concentration of bacteria, i.e. compounds do not show an inocculum effect, observed with other oral cephalosporins as described in R. C. Moellering Jr., “Antibiotics and Chemotherapy”, 47, 83-87, Karger, Basel (1995).

A selection of compounds I was highly stable in phosphate buffer pH 7.4 as well as in 0.01 N HCl solution. Moreover, a stability was observed in bovine blood serum corresponding to or higher than that of currently used oral cephalosporins.

In vivo antibacterial activity was demonstrated in an experimental systemic infection model with mice. The activity was higher than those with a clinically used combination of a penicillin and a β-lactamase inhibitor or an oral cephalosporin.

Therefore the present invention has the objective of providing a new class of cephalosporin antibiotics which is important in veterinary and human therapy and in inanimate systems. The high stability and broad spectrum antibacterial activity, even at high bacterial inoculi, of the compounds I according to the invention, in combination with their oral activity, could not be expected to this extent from the prior art.

The new compounds according to the invention are valuable antimicrobial substances which are active against Gram-positive and Gram-negative pathogens including also many penicillin- and cephalosporin resistant strains.

The free acid and in particular the alkaline and earth metal salts or the zwitterionic species are useful bactericides and can be empolyed to remove pathogens from dental and medical equipment for removing microorganisms and for therapeutic use in humans and animals. For this latter purpose, pharmaceutically acceptable salts as are known per se and are used in the administration of penicillins and cephalosporins, are used. These salts can be used together with pharmaceutically acceptable liquid and solid excipients to form suitable dose unit forms such as pills, tablets, capsules, suppositories, syrups, elixirs and the like, which can be prepared by processes which are known per se.

The new compounds are valuable antibiotics against many pathogenic penicillin sensitive or penicillin resistant bacteria and, accordingly, are useful in human and veterinary medicine. They can be used as antibacterial medicaments for treating infections caused by Gram-positive and Gram-negative bacteria, for example by Staphylococcus aureus, Escherichia coli, Enterococcus faecalis, Bacillus subtilis, Streptococcus pyogenes, Sreptococcus pneumoniae and Haemophilus influenzae.

The antibacterial agents can furthermore be used as additives for animal feeds, for preserving foodstuffs or feeds and as desinfectants. For example, they can be used in aqueous preparations in concentrations in the range 0.1 to 100 parts of antibiotic/million parts of solution for destroying and inhibiting the growth of harmful bacteria on medical equipment and as bactericides in industrial applications, for example in water-based paints and in soft water for paper mills, for inhibiting the growth of harmful bacteria.

The products according to the invention may be used alone or together with other active components in any of a large number of pharmaceutical preparations. These preparations can be used in capsule form or as tablets, powders or liquid solutions or as suspensions or elixirs. They can be administered orally, intravenously or intramuscularly.

The preparations are preferably administered in a form which is suitable for absorption through the gastrointestinal tract. Tablets and capsules for oral administration may be in dose unit form and can contain customary medicament excipients, such as binders, for example syrup, gum arabic, gelatin, sorbitol or polyvinylpyrrolidone, fillers, for example lactose, sugar, maize starch, calcium phosphate, sorbitol or glycine, lubricants, for example magnesium stearate, talc, polyethylene glycol or silica, disintegrants for example potato starch, or acceptable wetting agents such as sodium lauryl sulfate. The tablets may be coated by processes which are known per se. Oral liquid preparations can be in the form of of aqueous or oily suspensions, solutions, emulsions, syrups, elixirs and the like or can exist as dry product, for example for reconstitution before using water or other suitable excipients. Liquid preparations of this type can contain additives which are known per se, such as suspending agents, for example sorbitol syrup, methylcellulose, glucose/sugar syrup, gelatin, hydroxyethylcellulose, carboxymethylcellulose, aluminum stearate gel, or hydrogenated edible oils, for example almond oil, fractionated coconut oil, oily esters, propylene glycol or ethyl alcohol, preservatives, for example methyl or propyl p-hydroxybenzoate or sorbic acid. Suppositories contain suppository bases which are known per se, for example cocoa butter or other glycerides.

The preparations for injection can be in dose unit form in ampoules or in containers containing several doses along with an added preservative. The preparations can be in the form of suspensions, solutions or emulsions in oily or aqueous excipients, and they may contain formulation agents such as suspending agents, stabilizers and/or dispersants. Alternatively, the active component may be in powder form for reconstitution before using a suitable excipient, for example sterile, pyrogen-free water.

The preparations can also be in suitable form for absorption through the muscous membranes of the nose and of the throat or of the bronchial tissue, and can be in the form of powders or liquid sprays or inhalants, sucking sweets, as throat paints, etc.

For eye and ear medications, the preparations can be used in the form of individual capsules in liquid or semi-solid form or they can be used as drops, etc. Topical applications can exist or be formulated in hydrophobic vehicles as ointments, creams, lotions, paints, powders, etc.

The preparations according to the invention can contain, in addition to the excipient, other components such as stabilizers, binders, antioxidants, preservatives, lubricants, suspending agents, viscosity control agents or flavours or the like.

The preparations according to the invention may also contain, in addition to the excipient, other pharmacological agents, for examples, uricosurica, for example probenicid or β-lactamase inhibitors, for example clavulanate.

In addition, the preparations may contain one or more active antibacterial components to obtain a broader antibiotic range. Examples for such other active compounds are penicillins or aminogycosides.

For veterinary medicine, the preparations can be formulated, for example, as an intramammary preparation in either long-acting or rapid-release vehicles.

The dose to be administered is highly dependent on the state of the subject to be treated and the weight of the host, and on the method and frequency of administration. In general, a daily oral dose contains about 10 to about 120 mg of active component/kg of body weight of the subject in case of one or more administrations per day. A preferred daily dose for adult humans is in the range of about 20 to 80 mg of active component/kg of body weight.

The preparations according to the invention can be administered in various unit dose forms, for example in solid or liquid dose forms which can be taken orally. The preparations can contain 0.1 to 99% of active material per unit dose, either in solid or in liquid form. The preferred range is about 10 to 60%. The preparations generally contain 15 to about 1500 mg of active component but it is generally preferred to use a dose amount in the range about 250 to 1000 mg. In the case of parenteral administration, the unit dose is normally the pure compound in a sterile water solution or in the form of a soluble powder, which may be dissolved.

The examples below illustrate the products, processes, preparations and methods of treatment according to the invention.

### EXAMPLE 1

Preparation of diphenylmethyl (6R,7R)-(3-methoxymethylthio-8-oxo-7(-2-phenylacetamino)-5-thia-1-aza-bicyclo[4.2.0]oct-2-ene)-2-carboxylate

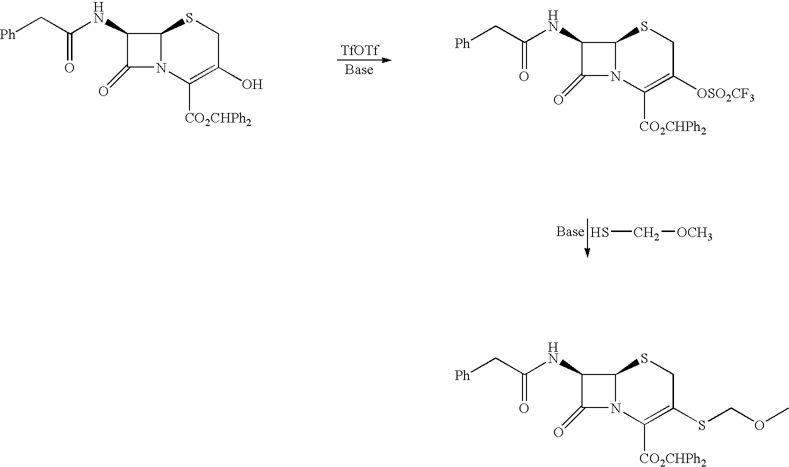

Diphenylmethyl (6R,7R)-8-oxo-7-phenylacetamino-3-trifluoromethylsulfonyloxy-5-thia-1-aza-bicyclo[4.2.0]oct-2-ene-2-carboxylate

In a three-necked flask fitted with a mechanical stirrer, a rubber septum and a balloon filled with dry nitrogen at −780 C to a solution of (6R,7R) diphenylmethyl 3-hydroxy-8-oxo-7-phenylacetamino-5-thia-1-aza-bicyclo[4.2.0]oct-2-ene-2-carboxylate (15.02 g, 30 mmol) in dry dichloromethane (300 ml) diisopropylethylamine (4.46 g, 34.5 mmol) was added and subsequently trifluoromethanesulfonic anhydride (9.73 g, 34.5 mmol) was added within 5 min with vigorous stirring. A beige precipitate was formed immediately. The suspension was stirred for additional 30 min at −78° C. The reaction suspension was diluted with dichloromethane (2500 ml) where upon a yellow solution was obtained. It was washed twice with portions of 10% aqueous sodium chloride solution. The combined aqueous layers were reextracted twice with portions (200 ml) of dichloromethane. The combined organic phases were dried over magnesium sulfate and the solvent removed in a vacuum rotary evaporator, leaving a beige solid. For purification the product was suspended in dry ether (300 ml). The solid was filtered and washed twice with potions of ether-pentane (1:1, 200 ml). After drying in high vacuum a colourless crystalline powder (18.28 g, 96%) was obtained, mp 211-212° C.

Diphenylmethyl (6R,7R)-(3-methoxymethylthio-8-oxo-7-(2-phenylacetamino)-5-thia-1-aza-bicyclo[4.2.0]oct-2-ene)-2-carboxylate

In a three-necked flask fitted with a mechanical stirrer, a dropping funnel and a balloon filled with nitrogen to a solution of the above-mentioned enol triflate (15.84 g, 25 mmol) in dry N,N-dimethylformamide (150 ml) at −78° C. dry dichloromethane (50 ml) was added and subsequently a solution (4.71 ml) of methoxymethanethiol (2.54 g, 32.5 mmol) in CDCl₃ added in one portion. To this mixture a solution of N,N-diisopropylamine (3.55 g, 27.5 mmol) in dichloromethane (125 ml) was slowly added over a 3.5 h period with vigorous stirring at −78° C. After the completed addion the reaction mixture was allowed to stir for additional 90 min at −78° C. until tic on silica gel indicated a complete reaction. The mixture was diluted with ethyl acetate (1000 ml) and subsequently washed with 1 N HCl (500 ml), 5% aqueous NaHCO₃ (500 ml) and 10% aqueous NaCl (500 ml). The organic phase was dried over magnesium sulfate and filtered. The resulting solution was evaporated in a vacuum rotary evaporator and the resuting oil dried in high vacuum, affording a pale yellow foam. For purification the crude product was washed with ether-pentane (1:1, 250 ml). After filtration and drying in high vacuum a pale yellow crystalline powder (11.28 g, 84%) was obtained, mp 159-161° C.

### EXAMPLE 2

Diphenylmethyl (6R,7R)-(8-oxo-7-phenoxyacetamino-3-trifluoromethylsulfonyloxy-5-thia-1-aza-bicyclo[4.2.0]oct-2-ene)-2-carboxylate and Diphenylmethyl (6R,7R) 3-methoxymethylthio-8-oxo-7-phenoxyacetamino-5-thia-1-aza-bicyclo[4.2.0]oct-2-ene-2-carboxylate (one pot procedure)

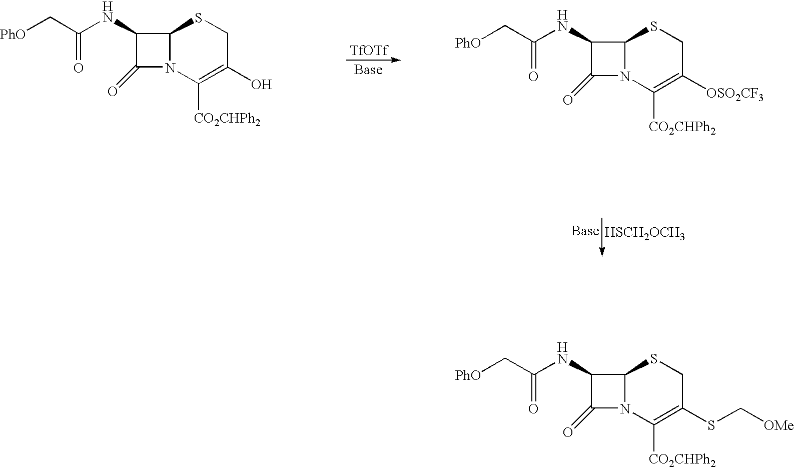

In a three-necked flask fitted with a magnectic stirrer, rubber septum and a balloon filled with dry nitrogen at −78° C. to a solution of (6R,7R) diphenylmethyl (3-hydroxy-8-oxo-7-phenoxyacetamino-5-thia-1-aza-bicyclo[4.2.0]oct-2-ene)-2-carboxylate (2.00 g, 3.87 mmol) in dry dichloromethane (30 ml) N,N-diisopropylethylamine (500 mg, 3.87 mmol) and subsequently trifluoromethanesulfonic anhydride (1.09 g, 3.87 mmol) were added with stirring where upon the reaction mixture turned light yellow. After 30 min at −78° C., tlc on silica gel indicated a complete reaction. To this solution at −78° C. a solution (8.48 ml) containing methyoxmethanethiol (393 mg, 5.03 mmol) in CDCl₃ was added. Subsequently, a solution of N,N-diisopropylethylemine (551 mg, 4.26 mmol) in dichloromethane (20 ml) was added slowly at −78° C. over a 3 h period. The reaction mixture was allowed to stir at −78° C. for additional 60 min and was then diluted with ethyl acetate (600 ml). The resulting solution was washed subsequently with 1 N HCl (300 ml), 5% NaHCO₃ (300 ml) and 10% NaCl (300 ml) and the organic layer dried over magnesium sulfate. After filtration and evaporation of the solvent in a vacuum rotary evaporator a pale beige noncrystalline solid (foam) resulted. The crude product was purified by recrystallisation from chloroform-hexane yielding (1.89 g, 81%). IR spectrum in ichloromethane: 3400, 2920, 1780, 1725, 1690, 1595, 1510, 1485, 1370, 1220, 1170, 1085 cm⁻¹.

### EXAMPLE 3

Diphenylmethyl (6R,7R)-(8-oxo-7-phenoxyacetamino-3-trifluoromethylsulfonvioxy-5-thia-1-aza-bicyclo[4.2.0]oct-2-ene)-2-carboxylate and diphenylmethyl (6R,7R)-3-acetaminomethylthio-8-oxo-7-phenoxyacetamino-5-thia-1-aza-bicyclo[4.2.0]oct-2-ene-2-carboxylate (one pot procedure)

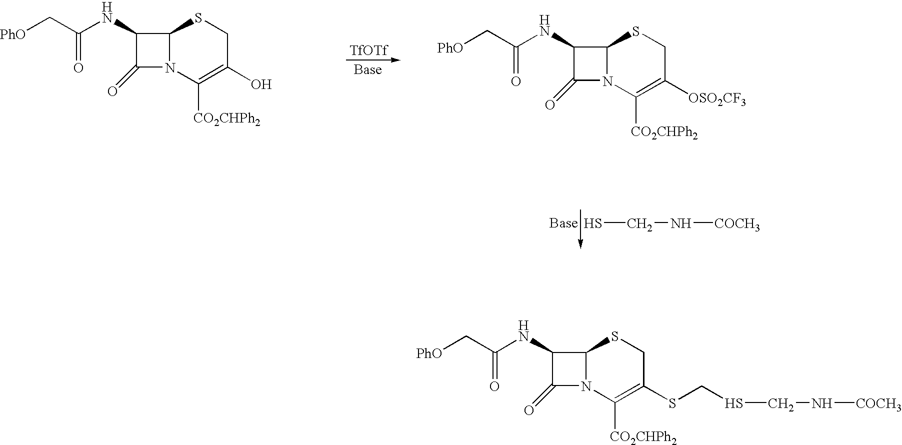

In a three-necked flask fitted with a magnectic stirrer, rubber septum and a balloon filled with dry nitrogen at −78° C. to a solution of (6R,7R) diphenylmethyl (3-hydroxy-8-oxo-7-phenoxyacetamino-5-thia-1-aza-bicyclo[4.2.0]oct-2-ene)-2-carboxylate (100 mg, 0.193 mmol) in dry dichloromethane (1.5 ml) N-Ethyl diisopropylamine (25 mg, 0.193 mmol) and subsequently trifluoromethanesulfonic anhydride (54.5 mg, 0.193 mmol) were added with stirring where upon the reaction mixture turned light yellow. After 30 min at −78° C., tic on silica gel indicated a complete reaction. To this solution at −78° C. a solution (0.50 ml) containing acetaminomethanethiol (105 mg, 0.25 mmol) in CDCl₃ was added. Subsequently, a solution of N,N-diisopropylethylamine (27.5 mg, 0.21 mmol) in dichloromethane (1.0 ml) was added slowly at −78° C. over a 3 h period. The reaction mixture was allowed to stir at −78° C. for additional 60 min and was then diluted with ethyl acetate (30 ml). The resulting solution was washed subsequently with 1 N HCl (15 ml), 5% NaHCO₃ (15 ml) and 10% NaCl (15 ml) and the organic layer dried over magnesium sulfate. After filtration and evaporation of the solvent in a vacuum rotary evaporator a pale beige noncrystalline solid (foam) resulted. It was purified by silica gel column chromatography using toluene-ethyl acetate (3:1) yielding a noncrystalline solid (55.9 mg, 52%). IR spectrum in dichloromethane: 3450, 2920, 1780, 1725, 1715, 1690, 1595, 1510, 1485 cm⁻¹.

### EXAMPLE 4

Preparation of diphenylmethyl (6R,7R)-(7-amino-3-methoxymethylthio-8-oxo-5-thia-1-aza-bicyclo[4.2.0]oct-2-ene)-2-carboxylate (removal of G side chain)

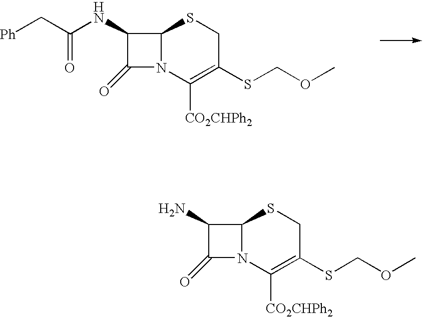

In a 50 ml Schlenk flask fitted with a rubber septum, magnet stirrer and a balloon filled with argon, at −20° C. to a solution of diphenylmethyl (6R,7R)-(3-methoxymethylthio-8-oxo-7-(2-phenylacetamino)-5-thia-1-aza-bicyclo[4.2.0]oct-2-ene)-2-carboxylate (2.80 g, 5 mmol) in dichloromethane (15 ml) is added N,N-dimethylaniline (1.50 g, 12.5 mmol) and subsequently, within 5 min, phosphorous pentachloride. The reaction mixture turned dark. Stirring was continued for 1.5 h at −15 to −20° C. Dry methanol (10 ml) was added at the same temperature and the mixture allowed to stir for additional 3.5 h. During this operation, a pale yellow precipitate was formed and the reaction mixture had disappeared. Ethyl acetate (300 ml) and water (150 ml) were added and the resulting mixture was stirred for 20 min. The pH of the aqueous phase was between 1 and 2. The organic phase was collected and the aqueous phase was extracted twice with portions (100 m) of ethyl acetate. The combined organic phases were washed with 10% sodium chloride (250 ml) and saturated sodium chloride (250 ml) and then dried over magnesium sulfate. After filtration, the solvent was removed in a vacuum rotary evaporator leaving a yellow oil. It was purified by chromatography using silica gel (30 g) with toluene-ethyl acetate (5:2, 20 fractions, 30 ml each). After removal of the solvent and drying in high vacuum a pale yellow non-crystalline solid (foam, 1.50 g, 68%) was obtained. IR spectrum in dichloromethane: 3400, 3330, 3030, 2960, 2930, 1775, 1725, 1600, 1490, 1445, 1365, 1220, 1180, 1130, 1085, 1010, 950 cm⁻¹. NMR-spectrum in CDCl₃: 1.89 (br s, 2H), 3.31 (s, 3H), 3.70 (d, 1H), 3.81 (d, 1H, J=17.4 Hz), 4.64 (d, 1H, J=12.2 Hz), 4.72 (d, 1H, J=4.9 Hz), 4.80 (d, 1H, 9H, J=12.2 Hz), 4.97 (d, 1H, J=4.9 Hz), 6.96 (s, 1H), 7.24-7.47 (m, 10H) ppm.

### EXAMPLE 5

Preparation of diphenylmethyl (6R,7R)-(7-amino-3-methoxymethylthio-8-oxo-5-thia-1-aza-bicyclo[4.2.0]oct-2-ene)-2-carboxylate (removal of V side chain)

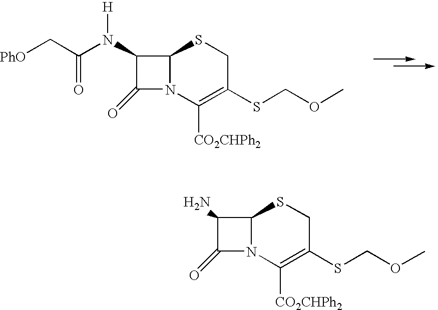

In a 50 ml Schlenk flask fitted with a rubber septum, magnetic stirrer and a balloon filled with argon, at −20° C. to a solution of diphenylmethyl (6R,7R)-(3-methoxymethylthio-8-oxo-7-(2-phenoxyacetamino)-5-thia-1-aza-bicyclo[4.2.0]oct-2-ene)-2-carboxylate (600 mg, 1.04 mmol) in dichloromethane (2 ml) is added N,N-dimethylaniline (315 mg, 2.6 mmol) and subsequently, within 5 min, phosphorous pentachloride (303 mg, 1.46 mmol). The reaction mixture was allowed to stir at −20° C. for 60 min. After 15 min a pale yellow precipitate had appeared. After 1 h of additional stirring a small sample was hydrolyzed for 15 min and the organic phase investigated by silica gel tic. A complete reaction was thus observed. The reaction mixture was then diluted with ethyl acetate (75 ml) and the resulting mixture stirred with water (35 ml) for 20 min. The pH of the aqueous solution was between 1 and 2. The organic phase was collected and the aqueous phase was extracted twice with portions (25 ml) of ethyl acetate. The combined organic phases were washed with 10% sodium chloride (50 ml) and saturated sodium chloride (50 ml) and then dried over magnesium sulfate. After filtration, the solvent was removed in a vacuum rotary evaporator leaving a yellow oil. It was purified by chromatography using silica gel (3.3 g) with toluene-ethyl acetate (5:2, 25 fractions, 3 ml each). After removal of the solvent and drying in high vacuum a pale yellow non-crystalline solid was obtained (331 mg, 72%). The spectral data were identical to those of product, obtained from G-side chain removal).

### EXAMPLE 6

Preparation of (6R,7R)-7-amino-3-methoxymethylthio-8-oxo-5-thia-1-aza-bicyclo[4.2.0]oct-2-ene-2-carboxylic acid

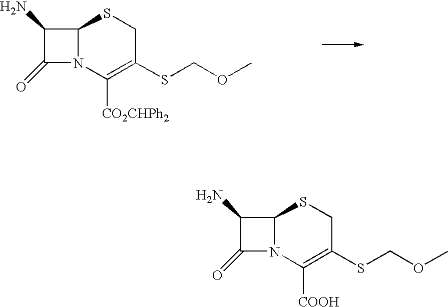

In a 10 ml Schlenk-flask fitted with a rubber septum, a magnetic stirrer and a ballon filled with argon, at 0° C., a solution of diphenylmethyl (6R,7R)-(7-amino-3-methoxymethylthio-8-oxo-5-thia-1-aza-bicyclo[4.2.0]oct-2-ene)-2-carboxylate (221.3 mg, 0.5 mmol) in methoxybenzene (2 ml) triflouroacetic acid (2 ml) was slowly added with stirring. After 3 h. The reaction mixture was slowly added with stirring to a mixture (40 ml) of pentane-ether (4:1) where upon a colourless precipitate was formed. It was collected by centrifugation and washed twice with portions (10 ml) of pentane-ether (4:1). The trifluoroacetic acid salt (110.4 mg, 80%) was dissolved in cold water (1.5 ml) and the pH adjusted to 7 with 10% sodium bicarbonate. Purification over HP-20 Diaion polymer resin (10 g, slow chromatography) with water (5 fractions, 10 ml each) water-methanol (4:1, 5 fractions 10 ml each) and water-methanol (3:2) yielded pure product (73 mg, 53%) after evaporation of the solvent in vacuum. The residue was dissolved in cold water (7 ml) and lyophilized at −20° C. to give pure product (73 mg, 53%). NMR in D₂O: 3.4 (s, 3H), 3.5 (dr 1H, J=18 Hz), 3.9 (d, 1H, J=18 Hz), 4.7(d, 1H, J=12 Hz), 4.9(d, J=12 Hz), 5.2 (d, J=4 Hz), 5.7(d, J=4 Hz).

### EXAMPLE 7

Preparation of (6R,7R)-(3-Methoxymethylmethylthio-8-oxo-7-(2-phenoxy-acetamino)-5-thia-1-aza-bicyclo[4.2.0]oct-2-ene)-2-carboxylic acid (1d) (by deprotection)

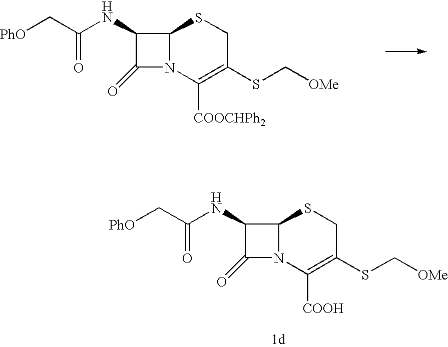

In 10 ml Schlenk flask, fitted with a rubber septum, a magnetic stirrer and an argon balloon, at 0° C., to a solution of diphenylmethyl (6R,7R) 3-methoxymethylthio-8-oxo-7-phenoxyacetamino-5-thia-1-aza-bicyclo[4.2.0]oct-2-ene-2-carboxylate (576 mg, 1 mmol) in methoxybenzene (5 ml) trifloroacetic acid (5 ml) is added slowly and the reaction mixture allowed to stir for 3.5 hr at 0° C. Tic on silica gel indicated a complete reaction. The reaction was slowly added at 0° C. to a pentane-ether (4:1, 100 ml) and the resulting precipitate collected by centrifugation. It was washed twice with portions (20 ml) of pentane-ether (4:1) affording the product as an amorphous powder (292 mg, 74%). NMR spectrum in CDCl₃/CD₃COOD: 3.4 (s, 3H) 3.7 (d, 1H, J=18 Hz), 3.9 (d, 1H, J=18 Hz), 4.6 (s, 2H), 4.85 (s, 2H), 4.8, 5.1 (d, 1H, J=4 Hz), 5.8 (dd, 1H, J=4 Hz, J=9 Hz), 6.9-7.3 (m, 5H), 7.4 (d, 1H, J=9 Hz).

### EXAMPLE 8

Preparation of (6R,7R)-(3-Acetminomethylthio-8-oxo-7-(2-phenoxyacetamino)-5-thia-1-aza-bicyclo[4.2.0]oct-2-ene)-2-carboxylic acid (1e) (by deprotection)

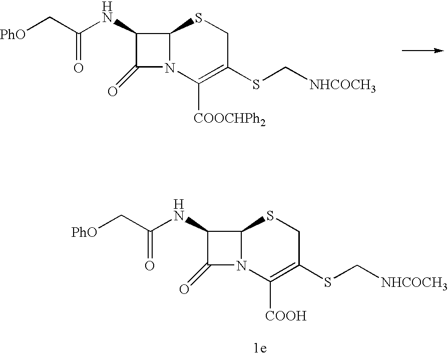

In a 5 ml Schlenk flask, fitted with a rubber septum, a magnetic stirrer and an argon balloon, at 0° C., to a solution of diphenylmethyl (6R,7R) 3-acetaminomethylthio-8-oxo-7-phenoxyacetamino-5-thia-1-aza-bicyclo[4.2.0]oct-2-ene-2-carboxylate (55.9 mg, 0.1 mmol) in methoxybenzene (0.5 ml) trifloroacetic acid (0.5 ml) is added slowly and the reaction mixture allowed to stir for 3.5 hr at 0° C. Tic on silica gel indicated a complete reaction. The reaction was slowly added at 0° C. to a pentane-ether (4:1, 10 ml) and the resulting precipitate collected by centrifugation. It was washed twice with portions (2 ml) of pentane-ether (4:1) affording the product as an amorphous powder (24 mg, 55%). NMR spectrum in D₂O/KHCO₃: 2.0 (s, 3H). 3.7 (d, 1H, J=18 Hz), 3.9 (d 1H, J=18 Hz), 4.6 (s, 2H), 4.4 and 4.6 (Abq 2H, J=14 Hz), 4.7 (d, 1H, J=12 Hz), 5.1 (d, 1H, J=4 Hz), 5.8(dd 1H, J=4 Hz, J=9 Hz), 5.8-6.2 (m, 5H).

### EXAMPLE 9

Preparation of (6R,7R)-(3-Methoxymethylthio-8-oxo-7-(2-phenoxyacetamino)-5-thia-1-aza-bicyclo[4.2.0]oct-2-ene)-2-carboxylic acid (1d) (by acylation)

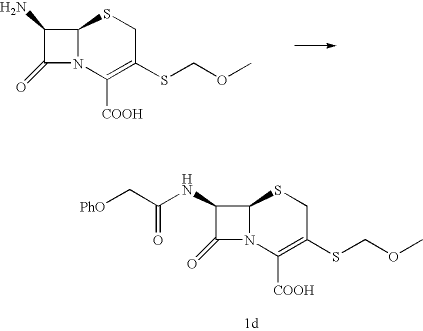

In a 10 ml Schlenk flask, fitted with a rubber septom, a magnetic stirrer and an argon balloon to a solution of (6R,7R)-7-amino-3-methoxymethylthio-8-oxo-5 thia-1-aza-bicyclo[4.2.0]oct-2-ene-2-carboxylic acid (138 mg, 0.5 mmol) in water (0.4 ml) and acetone (0.3 ml), containing sodium bicarbonate (140 mg, 1.66 mmol) at 0° C. with stirring a solution of phenoxyacetyl chloride (93 mg, 0.55 mmol) was added dropwise. The mixture was stirred at 5° C. overnight. Acetone was evaporated in vacuum and the aqueous solution was diluted with 2 ml of water and subsequently washed with ethyl acetate (2 ml) and then acidified to pH 2.5 in the cold and in the presence of ethyl acetate (5 ml). The organic layer was washed with cold water (2 ml) and the organic phase dried over magnesium sulfate. After filtration the solvent was removed in vacuum to give the product as a colourless solid (143 mg, 70%). The NMR-spectrum was identical to that of product from the deprotection procedure (Example 7).

### EXAMPLE 10

Preparation of potassium (6R,7R)-7-[(Z)-2-(2-amino-thiazol-4-yl)-2-methoximino-acetylamino]-3methoxymethylthio-8-oxo-5-thia-1-aza-bicyclo[4.2.0]oct-2-ene-2-carboxylate (1a)

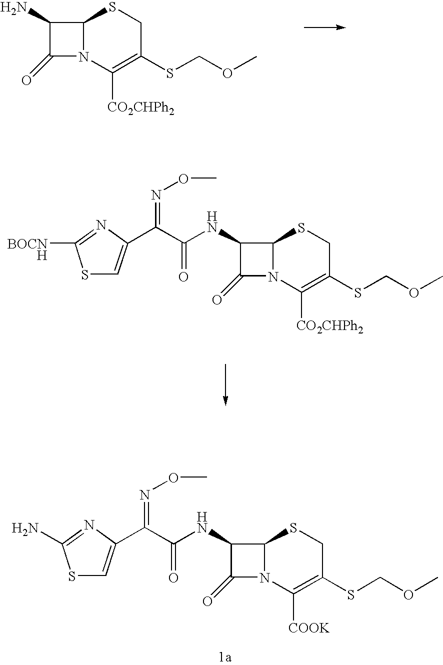

Diphenylmethyl (6R,7R)-7-[(Z)-2-(2-tert-Butoxycarbonylamino-thiazol-4-yl)-2-methoximino-acetylamino]-3-methoxymethylthio-8-oxo-5-thia-1-aza-bicyclo[4.2.0]oct-2-ene-2-carboxylate

In a 25 ml Schlenk flask, fitted with a rubber septum, a magnetic stirrer and a bollon filled with argon, at −20° C., to a suspension of diphenylmethyl (6R,7R)-(7-amino-3-methoxymethylthio-8-oxo-5-thia-1-aza-bicyclo[4.2.0]oct-2-ene)-2-carboxylate (200 mg, 0.45 mmol) and (Z)-(2-N-BOC-amino-thiazol4-yl)-2-methoxyiminoacetic acid (BOC-ATHMOA, 143 mg, 0.48 mmol) in dichloromethane (3.4 ml) N-methyl-morpholine (105 mg, 1.04 mmol) was added by a syringe. Stirring is continued until a clear solution is obtained (5 min). Phenylphosphoryl dichloride (114 mg, 0.54 mmol) is added dropwise and the reaction mixture allowed to stir for additional 45 min at −20° C. The mixture is diluted with ethyl acetate (15 ml) and then washed subsequently with 1N HCl (7 ml), 5% NaHCO₃ (7 ml), 10% NaCl (7 ml) and saturated NaCl (7 ml). The organic phase was dried over magnesium sulfate and, after filtration, the solvent was removed in a vacuum rotary evaporator. A yellow non-crystalline solid (foam) was obtained after short drying in high vacuum. It was purified by chromatography on silica gel (9 g) using toluene-ethyl acetate (7:1) and fractions (9 ml each). Evaporation of the solvent from fractions 6-19 and drying in high vacuum afforded the product as pale yellow non-crystalline solid (279 mg, 85%, foam). IR spectrum in dichloromethane: 3395, 3250, 3030, 2930, 1750, 1715, 1680, 1600, 1540, 1445, 1370, 1220, 1155, 1085, 1045, 1000, 950, 890 cm⁻¹.

Potassium (6R,7R)-7-[(Z)-2-(2-amino-thiazol-4-yl)-2-methoximino-acetylamino]-3-methoxymethylthio-8-oxo-5-thia-1-aza-bicyclo[4.2.0]oct-2-ene-2-carboxylate (1a)

In a dry Schlenk flask, fitted with a rubber septum, a magnetic stirrer and an argon balloon, at 0° C., to a solution of diphenylmethyl (6R,7R)-7-[(Z)-2-(2-tert-butoxycarbonylamino-thiazol4-yl)-2-methoximino-acetylamino]-3-methoxymethylthio-8-oxo-5-thia-1-aza-bicyclo[4.2.0]oct-2-ene-2-carboxylate (508 mg, 0.70 mmol) in methoxybenzene (3.5 ml) trifluoroacetic acid (3.5 ml) is slowly added. The mixture was allowed to stir at room temperature for 3.5 h, where upon tic on silica gel and reversed phase silica gel indicated a complete reaction. The solution was then dropwise added to a 4:1 mixture (70 ml) of pentane-ether. A beige solid precipitated. It was collected by centrifugation and washed twice with portions (40 ml) of pentane-ether (4:1). The crude product was suspended in cold water (14 ml) and the pH adjusted to 8 with solid KHCO₃, where upon the solid was dissolved. The solution was slowly chromatographed on HP-20-Diaion polymer resin (31 g) using water (7 fractions, 30 ml each) and water-methanol (4:1, 7 fractions, 30 ml each). The solvent was removed from fractions 8-13 in a rotary evaporator in high vacuum using a dry ice trap. After drying in high vacuum the residue was treated with dry ether to give a beige solid (220 mg, 63%), mp. 194-197° C. dec. NMR spectrum in D₂O: 3.41 (s, 3H), 3.63 (d, 1H, J=17.6 Hz), 3.91 (d, 1H, J=17.6 Hz), 4.00 (s, 3H), 4.76 (d, 1H, J=11.7 Hz), 4.94 (d, 1H, J=11.7 Hz), 5.27 (d, 1H, J=4.8 Hz), 5.80 (d, 1H, J=4.8 Hz), 7.04 (s, 1H).

### EXAMPLE 11

Preparation of potassium (6R,7R)-7-[(Z)-2-(2-amino-thiazol-4-yl)-2-hydroximino-acetylamino]-3-methoxymethylthio-8-oxo-5-thia-1-aza-bicyclo[4.2.0]oct-2-ene-2-carboxylate (1f)

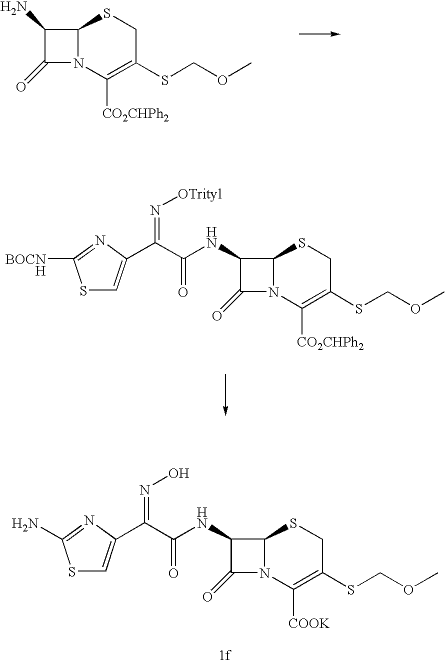

Diphenylmethyl (6R,7R)-7-[(Z)-2-(2-tert-Butoxycarbonylamino-thiazol-4-yl)-2-triphenylmethyloximino-acetylamino]-3-methoxymethylthio-8-oxo-5-thia-1-aza-bicyclo[4.2.0]oct-2-ene-2-carboxylate

In a 25 ml Schlenk flask, fitted with a rubber septum, a magnetic stirrer and a bollon filled with argon, at −20° C., to a suspension of diphenylmethyl (6R,7R)-(7-amino-3-methoxymethylthio-8-oxo-5-thia-1-aza-bicyclo[4.2.0]oct-2-ene)-2-carboxylate (100 mg, 0.225 mmol) and (Z(2-N-BOC-amino-thiazol-4-yl)-2-triphenylmethoxyiminoacetic acid (84.4 mg, 0.24 mmol)) in dichloromethane (1.7 ml) N-methyl-morpholine (52.5 mg, 0.52 mmol) was added by a syringe. Stirring is continued until a clear solution is obtained (5 min). Phenylphosphoryl dichloride (57 mg, 0.26 mmol) is added dropwise and the reaction mixture allowed to stir for additional 45 min at −20° C. The mixture is diluted with ethyl acetate (10 ml) and then washed subsequently with 1N HCl (5 ml), 5.% NaHCO₃ (5 ml), 10% NaCl (5 ml) and saturated NaCl (5 ml). The organic phase was dried over magnesium sulfate and, after filtration, the solvent was removed in a vacuum rotary evaporator. A yellow non-crystalline solid (foam) was obtained after short drying in high vacuum. It was purified by chromatography on silica gel (5 g) using toluenethyl acetate (19:1) affording the product as pale yellow non-crystalline solid (149 mg, 66%, foam). IR spectrum in dichloromethane: 3395, 3250, 3030, 2930, 1750, 1715, 1680, 1600, 1540, 1220, 1155, 1085 cm⁻¹.

Potassium (6R,7R)-7-[(Z)-2-(2-amino-thiazol-4-yl)-2-methoximino-acetylamino]-3-methoxymethylthio-8-oxo-5-thia-1-aza-bicyclo[4.2.0]oct-2-ene-2-carboxylate (1f)

In a dry Schlenk flask, fitted with a rubber septum, a magnetic stirrer and an argon balloon, at 0° C., to a solution of diphenylmethyl (6R,7R)-7-[(Z)-2-(2-tert-butoxycarbonylamino-thiazol-4-yl)-2-triphenylmethoximino-acetylamino]-3-methoxymethylthio-8-oxo-5-thia-1-aza-bicyclo[4.2.0]oct-2-ene-2-carboxylate (99.6 mg, 0.07 mmol) in methoxybenzene (0.35 ml) trifluoroacetic acid (0.35 ml) is slowly added. The mixture was allowed to stir at room temperature for 3.5 h, where upon tic on silica gel and reversed phase silica gel indicated a complete reaction. The solution was then dropwise added to a 4:1 mixture (7 ml) of pentane-ether. A beige solid precipitated. It was collected by centrifugation and washed twice with portions (7 ml) of pentane-ether (4:1). The crude product was suspended in cold water (7 ml) and the pH adjusted to 8 with solid KHCO₃, where upon the solid was dissolved. The solution was slowly chromatographed on HP-20-Diaion polymer resin (3 g) using water (7 fractions, 3 ml each) and water-methanol (4:1, 7 fractions, 3 ml each). The solvent was removed from fractions 8-12 in a rotary evaporator in high vacuum using a dry ice trap. After drying in high vacuum the residue was treated with dry ether to give a beige solid (22 mg, 64%). NMR spectrum in D₂O: 3.4 (s, 3H), 3.5 (d, 1H, J=17.6 Hz), 3.8 (d, 1H, J=17.6 Hz), 4.7 (d, 1H, J=11.7 Hz), 4.9 (d, 1H, J=11.7 Hz), 5.2 (d, 1H, J=4.8 Hz), 5.8(d, 1H, J=4.8 Hz), 7.0 (s, 1H).

### EXAMPLE 12

Preparation of dipotassium (6R,7R)-7-[(Z)-2-(2-amino-thiazol-4-yl)-2-carboxy-methoximino-acetylamino]-3-methoxymethylthio-8-oxo-5-thia-1-aza-bicyclo[4.2.0]oct-2-ene-2-carboxylate (1b)

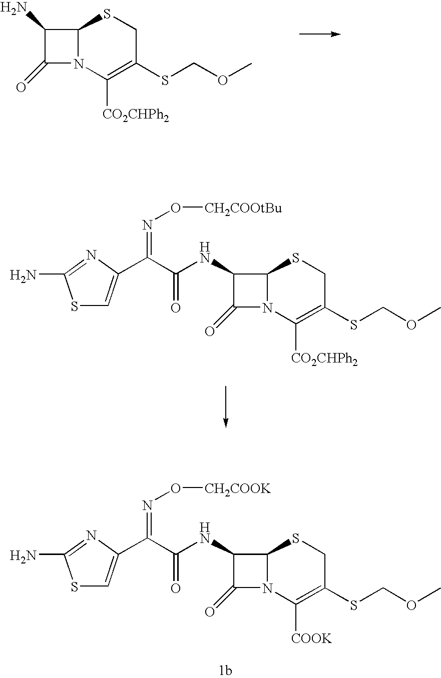

Diphenylmethyl (6R,7R)-7-[(Z)-2-(2-amino-thiazol)-2-(tert-butoxycarboxy-methoximino)-acetylamino]-3-methoxymethylthio-8-oxo-5-thia-1-aza-bicyclo[4.2.0]oct-2-ene-2-carboxylate

In a dry 25 ml Schlenk flask, fitted with a rubber septum a magnetic stirrer and a balloon filled with argon, at −20° C., to a suspension of diphenylmethyl (6R,7R)-(7-amino-3-methoxymethylthio-8-oxo-5-thia-1-aza-bicyclo[4.2.0]oct-2-ene)-2-carboxylate (443 mg, 1 mmol) and (Z)-2-(2-aminothiazolyl)-2-(tert-butoxlcarbonyl-methoxyimino)-acetic acid (ATMA) in dichloromethane (7.5 ml) N-methyl-morpholine (233 mg, 2.3 mmol) was added by a syringe. The mixture was allowed to stir for 10 min at −20° C., where upon a clear soltion resulted. Slowly, with stirring at −20° C., phenylphosphoryl dichloride (253 mg, 1.2 mmol) was added and the reaction mixture was stirred for 1.5 h at −20° C. It was then diluted with ethyl acetate (30 ml) and then washed subsequently with 1N HCl (15 ml), 5% NaHCO₃ (15 ml), 10% NaCl (15 ml) and saturated NaCl (15 ml). The organic phase was dried over magnesium sulfate and, after filtration, the solvent was removed in a vacuum rotary evaporator. A yellow non-crystalline solid (foam) was obtained after short drying in high vacuum. It was purified by chromatography on silica gel (18 g) using toluene-ethyl acetate (3:2) and taking fractions (18 ml each). Evaporation of the solvent from fractions 8-18 and drying in high vacuum afforded the product as pale yellow non-crystalline solid (573 mg, 79%, foam). IR spectrum in dichloromethane: 3480, 3380, 3250, 3030, 2970, 2930, 1780, 1720, 1680, 1600, 1530, 1450, 1375, 1220, 1145, 1120, 1085, 1040, 1000, 950, 890, 845, 810 cm⁻¹.

Dipotassium (6R,7R)-7-[(Z)-2-(2-amino-thiazol4-yl)-2-carboxy-methoximino-acetylamino]-3-methoxymethylthio-8-oxo-5-thia-1-aza-bicyclo[4.2.0]oct-2-ene-2-carboxylate

In a dry Schlenk flask, fitted with a rubber septum, a magnetic stirrer and an argon balloon, at 0° C., to a solution of diphenylmethyl (6R,7R)-7-[(Z)-2-(2-amino-thiazol4-yl)-2-(tert-butoxycarboxy-methoximino)-acetylamino]-3-methoxymethylthio-8-oxo-5-thia-1-aza-bicyclo[4.2.0]oct-2-ene-2-carboxylate (73 mg, 0.10 mmol) in methoxybenzene (0.5 ml) trifluoroacetic acid (0.5 ml) is slowly added. The mixture was allowed to stir at room temperature for 3.5 h, where upon tic on silica gel and reversed phase silica gel indicated a complete reaction. The solution was then dropwise added to a 4:1 mixture (10 ml) of pentane-ether. A beige solid precipitated. It was collected by centrifugation and washed twice with portions (7 ml) of pentane-ether (4:1). The crude product was suspended in cold water (3 ml) and the pH adjusted to 8 with solid KHCO₃, where upon the solid was dissolved. The solution was slowly chromatographed on HP-20-Diaion polymer resin (5 g) using water (10 fractions, 5 ml each). The solvent was removed from fractions 5-9 in a rotary evaporator in high vacuum using a dry ice trap. After drying in high vacuum the residue was treated with dry ether to give a beige solid (29 mg, 50%), mp. 196-198° C. dec. NMR spectrum in D₂O: 3.41 (s, 3H), 3.63 (d, 1H, J=17.6 Hz), 3.90 (d, 1H, J=17.6 Hz), 4.58(s, 2H), 4.76 (d, 1H, J=11.7 Hz), 4.95 (d, 1H, J=11.7 Hz), 5.27 (d, 1H, J=4.8 Hz), 5.82 (d, 1H, J=4.8 Hz) 7.05 (s, 1H).

### EXAMPLE 13

Preparation of (2′R,6R,7R)-7-(2′amino-2′-phenylacetamino)-3-methoxymethylthio-8-oxo-5-thia-1-aza-bicyclo[4.2.0]oct-2-ene-2-carboxylic acid (1c)

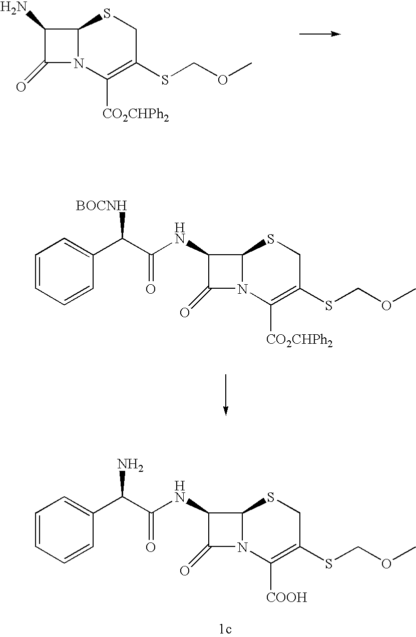

Diphenylmethyl (2′R,6R,7R)-7-(2T-tert-butyloxycarbonylamino-2′-phenylacetamino)-3-methoxymethylthio-8oxo-5-thia-1-aza-bicyclo[4.2.0]oct-2-ene-2-carboxylate

In a dry 10 ml Schlenk flask, fitted with a rubber septum, a magnetic stirrer and a balloon filled with argon, at −20 DC, to a suspension of diphenylmethyl (6R,7R)-(7-amino-3-methoxymethylthio-8-oxo-5-thia-1-aza-bicyclo[4.2.0]oct-2-ene)-2-carboxylate (100 mg, 0.23 mmol) and BOC-D-phenylglycine (60 mg, 0.24 mmol) in dichloromethane (1 ml) N-methyl-morpholine (233 mg, 2.3 mmol) was added by a syringe. The mixture was allowed to stir for 5 min at −20° C., where upon a clear soltion resulted. Slowly, with stirring at −20° C., phenylphosphoryl dichloride (57 mg. 0.27 mmol) was added and the reaction mixture was stirred for 30 min at −20° C. It was then diluted with ethyl acetate (10 ml) and then washed subsequently with 1 N HCl (5 ml), 5% NaHCO₃ (5 ml), 10% NaCl (5 ml) and saturated NaCl (5 ml). The organic phase was dried over magnesium sulfate and, after filtration, the solvent was removed in a vacuum rotary evaporator. A yellow noncrystalline solid (foam) was obtained after short drying in high vacuum. It was purified by chromatography on silica gel (6 g) using toluene-ethyl acetate (9:1) and taking fractions (6 ml each). Evaporation of the solvent from fractions 5-19 and drying in high vacuum afforded the product as pale yellow non-crystalline solid (573 mg. 79%, foam). IR spectrum in dichloromethane: 3400, 3400, 3020, 2960, 2920, 1780, 1720, 1685, 1595, 1495, 1365, 1310, 1215, 1160, 1110, 1080, 1025, 950 cm⁻¹.

(2′R,6R,7R)-7-(2′amino-2′-phenylacetamino)-3-methoxymethylthio-8-oxo-5-thia-1-aza-bicyclo[4.2.0]oct-2-ene-2-carboxylic acid

In a dry Schlenk flask, fitted with a rubber septum, a magnetic stirrer and an argon balloon, at 0° C., to a solution of diphenylmethyl (2′R,6R,7R)-7-(2′-tert-butyloxycarbonylamino-2′-phenylacetamino)-3-methoxymethylthio-8-oxo-5-thia-1-aza-bicyclo[4.2.0]oct-2-ene-2-carboxylate (67 mg, 0.10 mmol) in methoxybenzene (0.5 ml) trifluoroacetic acid (0.5 ml) is slowly added. The mixture was allowed to stir at room temperature for 1.5 h. The solution was then dropwise added to a 4:1 mixture (10 ml) of pentane-ether at 0° C. A beige solid precipitated. It was collected by centrifugation and washed twice with portions (5 ml) of pentane-ether (4:1). The crude product was suspended in cold water (3 ml). The solution was slowly chromatographed on HP-20-Diaion polymer resin (4.1 g) using water (5 fractions, 5 ml each), water-methanol (4:1) (5 fractions, 5 ml each) and water-methanol (3:2) (5 fractions, 5 ml each). The solvent was removed from fractions 6-13 in a rotary evaporator in high vacuum using a dry ice trap. After drying in high vacuum the residue was treated with dry ether to give a beige solid (25 mg, 61%), mp. 219-222° C. dec. NMR spectrum in D₂O: 3.37 (s, 3H), 3.43 (d, 1H, J=17.6 Hz), 3.75 (d, 1H, J=17.6 Hz), 4.58 (s, 2H), 4.71 (d, 1H, J=11.6 Hz), 4.88 (d, 1H, J=11.6 Hz), 5.11 (s, 1H), 5.12 (d, 1H, J=4.6 Hz), 5.69 (d, 1H, J=4.6 Hz) 7.42-7.54 (m, 5H).

### EXAMPLE 14

In Vitro Activity of Cephalosporins I

Minimal inhibitory cincentrations (MIC) were determined using 10 cm sterile plates using DIFCO Bacto Nutrient Agar (pH 6.8) with different concentrations of antibiotic (0.1 μg/ml up to 64 μl). The plates were then inoculated with a bacterial suspension obtained by diluting an overnight culture (37° C.) in DIFCO Bacto Nutrient Broth (pH 6.8) with sterile 1% NaCl (1:1000) using a 19 pin inoculator (Mast Diagnostics). MIC's (pig/ml) were determined 18 h after incubation at 37° C.

TABLE 1

| Bacterial strain, inoculum ca. 10⁴ | 1a | 1b | 1c |
|---|---|---|---|
| Staph. aureus DSM 1104 | 0.5 | 4 | 0.2 |
| Staph. aureus resistant | 2 | 16 | 2 |
| Staph. aureus 25768 | 16 | >64 | 8 |
| Escherichia coli DSM 1103 | 1 | 2 | 4 |
| Escherichia coli TEM 1 | 2 | 2 | >64 |

### EXAMPLE 15

Inoculum effect after inoculation with an non-diluted overnight culture (in comparison to Table 1). MIC's (μ/ml) were determined 18 h after incubation at 37° C.

TABLE 2

| Bacterial strain, inoculum ca. 10⁷ | 1a | 1b | 1c |
|---|---|---|---|
| Staph. aureus DSM 1104 | 0.5 | 4 | 0.5 |
| Staph. aureus resistant | 4 | 32 | 8 |
| Staph. aureus 25768 | 32 | >64 | 32 |
| Escherichia coli DSM 1103 | 1 | 2 | 4 |
| Escherichia coli TEM 1 | 2 | 2 | 32 |

### EXAMPLE 16

Chemical Stability of Cephalosporins I

The half-lives (h) of hydrolysis was determined at 37° C. by UV spectroscopy in physiological phosphate buffer and in physiological NaCl solution containing 0.01 N HCl.

TABLE 3

| pH | 1a | 1b | 1c |
|---|---|---|---|
| 7.4 | 405 | 419 | 181 |
| 2 | 375 | 398 | 171 |

### EXAMPLE 17

Blood Serum Stability of Cephalosporins I

The stability in blood serum was determined microbiologically using agar diffusion test with Escherichia coli TEM 1. The compounds I were incubated with sterile bovine serum (OXOID) at 37° C. At 0 h, 1 h, 2 h and 4 h intervals aliquot samples were spotted and remaining amounts of active antibiotic were calculated from the inhibition diameters (18-30 mm) in comparison with those (15-30 mm) obtained with 30 μg, 15 μg, 7.5μ and 3.75 μg of antibiotic.

TABLE 4

| | 1a | 1b |
|---|---|---|
| Serum half-life (37° C.) | 4.8 h | 2.7 h |

### EXAMPLE 18

In Vivo Activity of Cephalosporin 1a

In vivo activity (ED₅₀) was determined. Mice were infected intraperitoneally with ca. 10^{B} CFU of E. coli 3981 TEM-1. Untreated animals died within 48 h after infection. Compounds were administered by oral or subcutaneous route a first time 10 min after infection and a second time 4 h later. The 50% effective dose (ED₅₀) was calculated by the Spearman-Kärber method from the percentages of animals surviving to day 7 at each dose.

TABLE 5

| Antibiotic | Administration route | ED₅₀ (mg/kg) |
|---|---|---|
| 1a | Subcutaneous | 0.84 |
| 1a | Oral | 8.35 |

### EXAMPLE 19

Production of Pharmaceutical Preparations

A unit dose form is prepared by mixing 60 mg of potassium (6R,7R)-7-[(Z)-2-(2-amino-thiazol-4-yl)-2-methoximino-acetylamino]-3-methoxymethylthio-8-oxo-5-thia-1-aza-bicyclo[4.2.0]oct-2-ene-2-carboxylate with 20 mg of lactose and 5 mg of magnesium stearate and the 85 mg of mixture are added to a No. 3 gelatin capsule. Similarly, if more active constituents and less lactose are used, other dose forms may be prepared and filled into No. 3 gelatin capsules. Similarly, larger gelatin capsules and also compressed tablets and pills may also be produced. The following examples illustrate the production of pharmaceutical preparations.

Tablet (For Oral Application)

| | |
|---|---|
| Potassium (6R,7R)-7-[(Z)-2-(2-amino-thiazol-4-yl)-2- | 120 mg |
| methoximino-acetylamino]-3-methoxymethylthio-8-oxo-5- | |
| thia-1-aza-bicyclo[4.2.0]oct-2-ene-2-carboxylate | |
| Corn starch | 6 mg |
| Magnesium stearate | 232 mg |
| Dicalcium phosphate | 192 mg |
| Lactose | 250 mg |

The active constituent is mixed with the dicalcium phosphate, lactose and about half of the corn starch and coarse-sieved. It is dried in high vacuum and again sieved through sieves having mesh widths of 1.00 mm (No. 16 screens). The rest of the corn starch and the magnesium stearate is added and the mixture is pressed to give tablets which each weight 800 mg and have a diameter of about 1.27 cm (0.5 in.).

Parenteral Solution

| | |
|---|---|
| Ampoule | |
| Potassium (6R,7R)-7-[(Z)-2-(2-amino-thiazol-4-yl)-2- | 250 mg |
| methoximino-acetylamino]-3-methoxymethylthio-8-oxo-5- | |
| thia-1-aza-bicyclo[4.2.0]oct-2-ene-2-carboxylate | |
| Sterile water (is added from a separate ampoule | 4 ml |
| using a syringe immediately before use) | |

Ophtalmic Solution

| | |
|---|---|
| Potassium (6R,7R)-7-[(Z)-2-(2-amino-thiazol-4-yl)-2- | 50 mg |
| methoximino-acetylamino]-3-methoxymethylthio-8-oxo-5- | |
| thia-1-aza-bicyclo[4.2.0]oct-2-ene-2-carboxylate | |
| Hydroxypropylmethylcellulose | 5 mg |
| Sterile water (is added from a separate ampoule using a | 1 ml |
| syringe immediately a syringe immediately before use) | |

Otic Solution

| | |
|---|---|
| Potassium (6R,7R)-7-[(Z)-2-(2-amino-thiazol-4-yl)-2- | 50 mg |
| methoximino-acetylamino]-3-methoxymethylthio-8-oxo-5- | |
| thia-1-aza-bicyclo[4.2.0]oct-2-ene-2-carboxylate | |
| Benzalkonium chloride | 0.1 mg |
| Sterile water (is added from a separate ampoule using a | 1 ml |
| syringe immediately a syringe immediately before use) | |

Topical Cream or Ointment

| | |
|---|---|
| Potassium (6R,7R)-7-[(Z)-2-(2-amino-thiazol-4-yl)-2- | 100 mg |
| methoximino-acetylamino]-3-methoxymethylthio-8-oxo-5- | |
| thia-1-aza-bicyclo[4.2.0]oct-2-ene-2-carboxylate | |
| Polyethylene glycol 4000 | 400 mg |
| Polyethylene glycol 400 | 1.0 g |

The active component in the above preparations can be mixed alone or together with other biologically active components, for example with other antibacterial agents such as a penicillin or cephalosporins or with other therapeutic agents, such as probenicid.

It is understood that the specification and examples are illustrative but not limitative of the present invention and that other embodiments within the spirit and scope of the invention will suggest themselves to those skilled in the art.

1. A Compound of the structural formula I 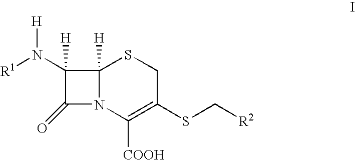
or a pharmaceutically acceptable salt, ester or amide derivative thereof wherein R¹ denotes a pharmaceutically acceptable side chain radical as used conventionally in the field of cephalosporins and wherein R² denotes a pharmaceutically acceptable group which is bonded to the remaining part of the molecule by an oxygen-carbon single bond or a nitrogen carbon single bond.

2. A compound according to claim 1, caracterized in that R¹ denotes a pharmaceutically acceptable side chain radical selected from phenylacetyl, phenoxyacetyl, 2-amino-2-phenylacetyl, 2-amino-2-(4-hydroxyphenyl)acetyl, 2-amino-2-(1,4-cyclohexadienyl)acetyl, 2-hydroxy-2-phenylacetyl, 2-hydroxy-2-(4-hydroxyphenyl)acetyl, Z-2-(2-amino-4-thiazolyl)-2-(methoximino)acetyl, Z-2-(2-amino-4-thiazolyl)-2.2-difluoromethoximino)-acetyl, Z-2-(2-amino-4-thiazolyl)-2-(carboxymethoxyimino)acetyl, Z-2-(2-aminothiazolyl)-2-((1-carboxy-1-(methylethoxy)imino)acetyl, Z-2-(2-aminofthiazolyl)-2-(hydroxyimino)acetyl, Z-2-(2-aminothiazol-4-yl)-2-pentenoyl, 2-(2-amino-4-thiazolyl)-4-carboxy-1-oxo-2-butenyl, 2-(cyanomethylthio)acetyl, 2-(difluoromethylthio)acetyl, 2-(fluoromethylthio)acetyl, 2-(2-amino-2-carboxyethylthio)acetyl, α-(4-ethyl-2,3-dioxo-1-piperazinecarboxamid)-α-4(4-hydroxyphenyl)acetyl, 2-(2-(aminomethyl)phenyl)acetyl, [4-(2-amino-1-carboxy-2-oxoethylidene)-1,3-dietan-2-yl]carbonyl, 2-thienylacetyl, 1-(1H)-tetrazolylacetyl, 2-(3,5-dichloro-4-pyridon-1-yl)acetyl, 2-(5-carboxy-4-imidazolylcarboxamido)phenylacetyl, phenylsulfoacetyl, 2-furanyl(methoxyimino)acetyl, cyanoacetyl, 5-amino-5-carboxy-1-oxopentyl, 2-(4-pyridylthio)acetyl, 5-amino-1,2,4-thiadiazol-3-yl(methoximino)acetyl, 1H-pyrazol-3-yl(methoximino)acetyl, and wherein R² denotes a pharmaceutically acceptable group which is bonded to the remaining part of the molecule by an oxygen-carbon single bond or a nitrogen-carbon single bond and which is selected from the group comprising substituted or unsubstituted: alkoxy, alkenyloxy, alkinyloxy, cycloalkoxy, N-heterocyclyl, heterocyclyloxy, heterocyclylcarbonyloxy, heterocyclylthiocarbonyloxy, acyloxy, thioacyloxy, alkoxycarbonyloxy, carbamoyloxy, thiocarbamoyloxy, heterocyclyloxycarbonyloxy, heterocyclyloxythiocarbonyloxy, N-heterocyclycarbamoyloxy, N-heterocyclylthiocarbamoyloxy, heterocyclylcarbonylamino, heterocyclylthiocarbonylamino, heterocyclyloxycarbonylamino, acylamino, alkoxycarbonylamino, alkoxythiocarbonylamino, thioacyclamino, N-heterocyclylcarbamoylamino, N-heterocyclylthiocarbamoylamino, carbamoylamino, thiocarbamoylamino, imidoylamino, guanidino, N-heterocyclyl-alkoxycarbonylamino, N-heterocyclyl-alkylthiocarbonylamino and N-sulfonylamino where the foregoing alkyl, alkenyl, alkinyl, acyl, thioacyl or imidoyl molecule parts contain 1 to 6 carbon atoms and the heterocyclyl moiety is monocyclic or bicyclic and contains 3 to 10 ring atoms, of which one or more are selected from the series comprising: oxygen, sulphur or nitrogen and where the substituents of the above-mentioned groups R², independently of one another, may be: alkyl, acyl, thioacyl, heterocyclyl, hydroxyl, hydroxyalkyl, alkoxy, hydroxyalkoxy, aminoalkoxy, amidinoalkoxy, guanidinoalkoxy, acyloxy, heterocyclyloxy, alkylheterocyclyloxy, hydroxyalkylheterocyclyloxy, aminoalkylheterocyclyloxy, carbamoyl, alkylcarbamoyl, dialkylcarbamoyl, carbamoyloxy, alkylcarbamoyloxy, dialkylcarbamoyloxy, thiocarbamoyl, alkylthiocarbamoyl, dialkylthiocarbamoyl, thiocarbamoyloxy, alkylthiocarbamoyloxy, dialkylthiocarbamoyloxy, mercapto, alkylthio, hydroxyalkylthio, aminoalkylthio, monoalkylaminoalkylthio, dialkylaminoalkylthio, amidinoalkylthio, acylthio, heterocyclylthio, alkylheterocyclylthio, hydroxyalkylheterocyclylthio, aminoalkylheterocyclylthio, carbamoylthio, monoalkylcarbamoylthio, dialkylcarbamoylthio, thiocarbamoylthio, alkylthiocarbamoylthio, dialkylcarbamoylhio, amino, monoalkylamino, hydroxyalkylamino, aminoalkylamino, dialkylamino, oxo, oximino, or alkylimino, imidoylamino, alkylimidoylamino, dialkylimidoylamino, tetraalkylammonium, cycloalkylamino, heterocyclylamino, alkylheterocyclylamino, heterocyclylcarbonylamino, alkylheterocyclylcarbonylamino, acylamino, amidino, monoalkylamidino, dialkylamidino, guanidino, alkylguanidino, dialkylguanidino, carbamoylamino, thiocarbamoylamino, alkylcarbamoylamino, thiocarbamoylamino, alkylthiocarbamoylamino, nitro, chloro, bromo, fluoro, iodio, azido, cyano, alkylsulphinyl, alkylsulphonyl, sulphonamido, sulphamoyloxy, alkylsulphamoyloxy, alkylsulphonyloxy or sulpho, sulphoxy, carboxamido, N-monoalkylcarboxamido, N,N-dialkylcarboxamido or carboxy, where the substituents, independently of one another, occur once or several times and their alkyl moiety contains 1 to 6 carbon atoms, and where the heterocyclic moiety is monocyclic or bicyclic and contains 3 to 10 ring atoms, of which one or more are selected from the series comprising: oxygen, sulphur and nitrogen and its pharmaceutically acceptable salts esters and amide derivatives.

3. A compound according to claims 1 and 2, characterized in that R¹ is defined as in claim 2 and wherein R² denotes a pharmaceutically acceptable group which is selected from methoxy or acetylamino and ist salts, esters and amide derivatives.

4. An antibacterial composition comprising an antibacterially effective amount of a compound according to claims 1 to 3 and a pharmaceutical excipient therefor.

5. A compound according to claims 1 to 3 in unit dose form.

6. A process for preparing the compounds according to claims 1 to 3, which comprises reacting, compound of formula 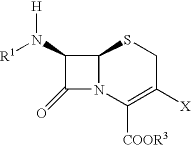
wherein R¹ and R² are as defined in claims 1 to 3, is a conventional leaving group and R³ is hydrogen or a conventional carboxy protecting group with a thiol of formula
HS—CH₂—R²
or an organic or inorganic salt thereof.

7. A process for preparing the compounds according to claims 1 to 3 which comprises reacting a compound of formula 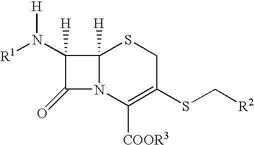
wherein R¹ is as defined in claims 1 to 3, R² is hydrogen and R³ is a conventional carboxy protection group or hydrogen, with an acylating agent of formula
R¹—X
wherein R¹ is as defined in claim 1 to 3 and X is a conventional leaving group.

8. A process for preparing the compounds according to claims 1 to 3 which comprises reacting with a deprotection agent a compound of formula 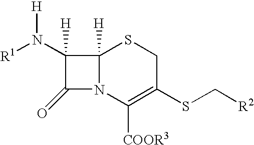
wherein R¹ and R² are as defined in claims 1 to 3 and R³ is a conventional carboxy protection group.

9. An N-deacylation process leading to an intermediate in the preparation of compounds according to claims 1 to 3 caracterized in that a compound of of formula 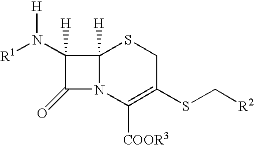
wherein R² is as defined in claims 1 tp 3, R¹ is phenylacetyl or phenoxyacetyl and R³ is a conventional carboxy protection group, is reacted subsequently with an inorganic acid chloride, a C₁ to C₆ alcohol and water. ×10. Intermediate for the preparation of compounds according to claims 1 to 3 of the formula 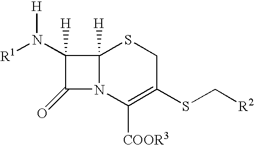
wherein R² is as defined in claims 1 to 3, R¹ is hydrogen and R³ is hydrogen or a conventional carboxy protecting group.
